(19) United States
(12) Patent Application Publication
(10) Pub. No.: US 2009/0074262 A1
(43) Pub. Date: Mar. 19, 2009

(54) MEDICAL INJECTOR WITH AUTOMATIC DOSAGE DETERMINATION AND ELECTRONIC MEANS FOR AUTHENTICATION OF USER

(75) Inventor: Srinivas Rao Kudavelly, Hyderabad (IN)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/299,123

(22) PCT Filed: Apr. 27, 2007
(86) PCT No.: PCT/IB07/51575

§ 371 Date: Oct. 31, 2008

(30) Foreign Application Priority Data
May 4, 2006 (EP) 06113485.4

(51) Int. Cl. A61M 5/31; G06K 9/00

(52) U.S. Cl. 382/124; 604/189

(57) ABSTRACT

The invention relates to a medical injector, especially a syringe, with means (4, 5, S) for automatic dosage determination. It is the object of the invention to prevent unauthorized usage of medical injectors. Therefore the medical injector is applied with further electronic means for authentication (3) of user and/or medical staff.

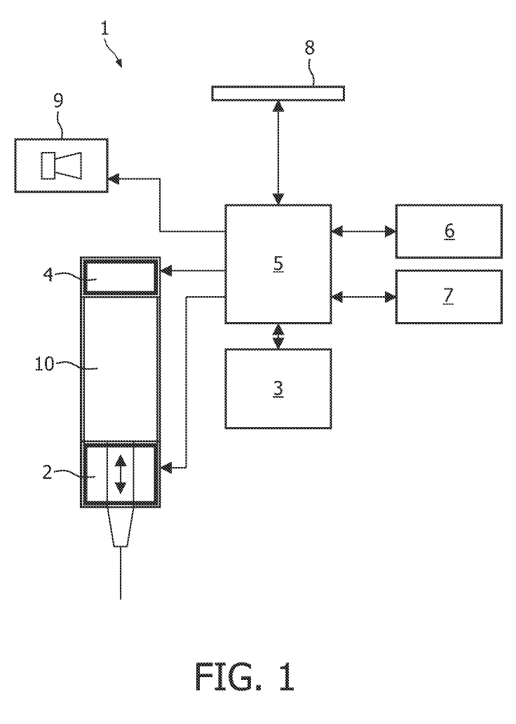

The invention relates to a medical injector, especially a syringe, with means for automatic dosage determination.

The problem in usage of injectors or syringes is, that in the use of unauthorized persons it can cause most critical damage to human health.

The possibility of virus transmission like HIV, Hepatitis, and so on is only one problem, which is caused by unauthorized usage of injectors.

Another relevant problem is drug abuse. A high quantity of HIV and Hepatitis C infections are caused by unauthorized usage of infected, not clean injectors.

The EP 1 498 067 A1 discloses a dosage determination supporting device especially for the usage for insulin medication. This injector is only applied for a special use for insulin therapy, by a unit which calculates to an actual blood glucose concentration an appropriate and fitted amount of insulin, which has to be injected.

This kind of syringe offers more functionality than usual syringes, but the injection can be made by any person, skilled in the use of this electronic syringe. Nevertheless, this is not a criteria for an authorized use of it.

It is an object of the invention to prevent unauthorized usage of medical injectors.

It is also an object of the invention to realize this with means, which are so compact and easy to be produced in mass.

Therefore, a medical injector, especially a syringe, is supported with means for dosage determination, and further electronic means for authentication of user and/or medical staff.

Last feature is most important in combination with the dosage means.

First, the authorized usage of the syringe is guaranteed by doubtless authentication of the user. Only by this, a dosage of pharmaceuticals is possible.

Another advantage of the combination of these features is, that not only authorized usage is given, but also the automatic dosage of pharmaceuticals to an individual patient.

A first embodiment of the invention is supported by electronic means for authentication which are implemented directly in the medical injector unit. So this combination of features realizes a compact and intelligent unit.

A further embodiment of the invention is to apply a biometric sensor for authentication which is directly incorporated in the injector or corresponding with the injector. By this, authentication can be realized effectively.

By a further embodiment data storage means and steering means are applied for patient relevant dosage of pharmaceuticals. So the unit carries the individual patient relevant dosage data on one side, and the authorisation of the person who is allowed to apply this pharmaceutical to the patient on the other side.

By further embodiment according to the aforesaid feature combination it is very advantageous, that the data storage means are applied to store usage-history data of the injector, in order to store medicals or drug dosage of usage-history data in context with a patient, who was supplied with it. The terms drug and medical are used synonym in this description.

To guarantee a high security standard in the sense of the invention, a further embodiment is, that the needle of the injector is supplied by retraction means, in order to retract the needle before or after usage, by which the needle comes out automatically after valid authentication. So not only the action of injection is necessary to be authorised, but also the use or the impact of the needle is to be refused, if there is no authentication. So the syringe is very effective in prevention of the use of unauthorized persons, in order to prevent the transmission of HIV or Hepatitis and so on.

A very effective and quick, but easy technical way for authentication is given by a further embodiment of the invention, by which a biometric sensor like a finger print reading sensor is applied. A finger print is a very individual signification of a person. So this is an easy way for authentication.

For higher security standard, an alternative embodiment uses a code generating interface for authentication, for example a number terminal.

Very advantageous is an embodiment, in which the medical injector is a common portable unit, for example a common syringe equipped with an electronic means for authentication.

For more complex use, for example in hospital, an alternative embodiment is a medical injector as a stationary unit. This unit can be connected to further medical apparatus, for example for complex medical operation.

A further embodiment of the invention is, to apply a data transfer and/or programming interface into the injector unit. By this, the injector or the syringe can be programmed by a computer in which personal health data are stored, or the injector can be driven by a specialist who is located at another place, however for example involved into an operation via data communication or internet communication. Moreover, this feature of data transfer empowers home therapy of a patient at home and medical staff doing the treatment remote from patient at hospital or medical practice.

A further advantageous embodiment is that the expiry date of the medicals can be programmed in that way, that after expiry date, the injector needle does not retract or come out, so that the injector can not be used. By this inventive injector a high security standard is realised. So, it is absolutely prevented, that the patient receives medicals or drugs which have passed the expiration date. In most cases such medicals can initiate serious illness. By this feature, this misuse can completely be excluded.

A further advantageous embodiment is, that the injector is also embodied with a data or data network transfer interface with a two-way-data-transfer in order to communicate use data as well as therapy and patient data internally and externally with a computer or computer system or computer network. Patient data can be stored in data storages, which are implemented in the injector unit, or they can be stored in external data storages. In the last case, the injector unit automatically corresponds with external computers to order specific patient data, which are given free for this special use.

Furthermore, it is possible to use both, internal data storage as well as external data storage, which can be exchanged in both directions. So in result is created an intelligent injector, which is able to correspond in both directions, in order to send information, but also to receive new information or functional order.

This special injector is able to be used also in everyday clinical life al well as for example in remote surgery or internet supported surgery. External specialist/physician for example can cause specially focused injection via internet, or can send special injection parameter directly to the two-way-corresponded injector.

A further embodiment with a high functional utility value is, that the injector is applied with internal storage means or can correspond with external storage means, in order to result validation of use, or will give actual usage information.

A further advantageous embodiment for a safe use is, that patient allergy data for various medicals is stored internally or externally in that way, that the injector will be deactivated automatically, when the drug given is not suitable with the patient. Allergy reaction is one of the most dangerous situations which can occur during medication of drugs. For this, the correspondence of physical use validation of the injector and the allergy data of the patient is very important. By this kind of automatic validation of the injector, it causes a very safe use for the patient as well as for the physician.

A further embodiment with a high practical aspect in use is that if the current drug reaches a programmable filling state mark, for example almost nearly completion, then a signal generates automatically an order of newer drug, if authorized by the physician. This is of a high practical aspect to optimize the usage in daily hospital use, but also to care for refill just in time when it must be available for use.

An advantageous embodiment is to implement a timer and/or an alarm facility into the injector unit. By this measurement an unauthorized use of the syringe or the injector is marked acoustically.

If a timer is implemented, like mentioned in the aforesaid embodiment, it is advantageous to apply the timer with an Auto-Off-function, in order to switch off the injector and/or retract the needle: If the validation of authentication will not be given in an adjustable time limit Auto-Off function is also triggered in the situation, if the user does not inject the medical in the adjustable time limit, so the needle will be retracted too. As a conclusion the unauthorized usage is strictly blocked in a technical and therefore automatically way. If somebody who is not authorized would try to use the injector, the injector refuses the injection or retracts the needle. By this measurement a high standard of security is realized.

A last, but regarding compactness an important embodiment is to implement a compact lithium cell into the injector, or injector-unit. Thereby a long time independent use of the injector is possible.

So the advantages of the invention in sum are.

Drug abusers will not be able to use the thrown or used syringes which are not disposed properly, as it is equipped with auto off feature wherein after a specific time, the syringe needle retracts back. Also as the syringe can be equipped with biometric validation, it can not be used by any other user apart from the intended user.

For diabetic patients, especially aged people, wherein diabetes is very predominant, can take this drug unassisted without worrying about the dosage and retrieval of the insulin from the bottle through syringe. The biometric syringe contains the insulin a-priori and it is pre-programmed to inject the specific amount of the drug at any instant of operation, by this way it prevents the inadvertent injection of less or more drug. Also the biometric syringe is equipped with a beeper generating an acoustic signal, which will aid the user during the entire process.

In most cases, the diabetic patient needs to take the drug at regular periods, the biometric syringe is equipped with alarm which can be programmed so that it can inform/alert the user about the impeding usage. Even if the user forgets and tries to take the drug before it is due, the biometric syringe can not be activated.

For an anaesthetist, the biometric syringe prevents errors during an operation in situations where danger of drug injection in excess exists. In case of biometric syringe the anaesthetist can pre-program the drug dosage.

An embodiment of the invention is shown in the drawing. The embodiment is only for exemplary description and does not delimit the scope of the invention which is given by the independent claim.

The drawing shows the features of an embodiment of the invention in principle.

A syringe, which represents an injector unit in general shows a needle which can be retracted by retraction means 2 into the inner part of the syringe 1, so that the syringe is prevented to be used. Dosage means 4 like a micro pump are applied into the syringe 1 in order to press out a defined amount of pharmaceutical. The dosage means 4 and the retraction means 2 are steered by steering means 5.

The steering means 5 are logically connected with data storage means 6, a timer 7 and an authentication unit 3. The injector is functionally secured electronically in this way. A first initial step before usage is to give authentication of the user or patient by finger print or something else, which guarantees a secure usage and authorization. Then after authentication, the authentication unit 3 sends a signal to enable the usage of the injector. So first, the needle will be driven out of the retracted position into usage position whereby the needle is extended, as shown in the FIGURE.

Further intercommunication between data storage means 6 and steering unit gives free usage of for example predetermined amounts of pharmaceutical to be injected, concerning to patient and/or the user. So dosage data and also historical data of former medication which is relevant to the authorized person or medical staff, will be communicated to the steering means 5, so that inside the steering means 5 the signal for the dosage pump 4 can be generated.

This data will also be correlated to timer data, so that historical data can be stored. Also, timer function for duration can be supplied by this. The time scales of the timer 7 are adjustable, so that operation times or other time dependent functions can be adjusted.

The steering means 5 are also connected with an acoustic alarm generator 9. If usage from a person not authorized is tempted, the alarm generator will be generated automatically if operation of the injector would be tried without authentication, or even if the pharmaceutical is running empty, or in case of malfunction, or even to remember the user to inject the medical.

The steering means 4 are also logically connected with a data interface. By this the injector unit can communicate with one or more computers at other places. This is very advantageous, if medical staff or a specialist at another place is involved in actual operation or therapy via remote communication or internet.

By this, the injector can be driven via terminal at any place on the world. This would be advantageous during harmful operation in cancer therapy, or where expertise for surgical operation is not available locally and support of an expert available at another place is needed. So by an expert residing at any place on the world remote from the patient, a specific injection can be caused. This could be advantageous during surgical operation, if local injections of strong cancer pharmaceuticals must be applied to the patient by experts, for example.

On the other hand, this interface can also be for example a blue tooth or an infrared interface, by which the injector unit will be steered and supplied by patient data, which are asked for to be transmitted to the injectors steering unit from a central medical computer system.

By all these features a very secure usage of the injector is realized. Drug abuse can be excluded completely.

The components of the injector disclosed in this description can be separated physically, and the data transfer between the components as shown in the drawing is realized.

Nevertheless, the injector components can also be sampled in a common injector or syringe unit. The used technique is a microelectronic technique, in which actors like the needle retractor 2 and the dosage pump 4 can be realized by micromechanical means, for example on the basis of semiconductor techniques. Compact lithium cells can be used, which can also be used in a compact injector unit.

1. A medical injector, especially a syringe, with means (4, 5, 6) for dosage determination, and further electronic means for authentication (3) of a user and/or medical staff.

2. A medical injector according to claim 1, characterized in that the electronic means for authentication (3) are implemented directly in the medical injector unit.

3. A medical injector according to claim 1, characterized in that the electronic means for authentication (3) comprise a biometric sensor which is directly incorporated in the injector or corresponding with the injector.

4. A medical injector according to claim 1, characterized in that data storage means (6) and steering means (5) are applied for patient dependent dosage of medicals.

5. A medical injector according to claim 4, characterized in that data storage means (6) are applied to store usage-history data of the injector, in order to store medicals dosage of usage-history data in context with a patient, who was supplied with it.

6. A medical injector according to claim 4, characterized in that the expiry date of the drugs can be programmed in that way, that after expiry date, the injector needle will not retract or come out, so that the injector can not be used.

7. A medical injector according to claim 1, characterized in that the needle of the injector is supplied by retraction means (2), in order to retract the needle before or after usage, by which the needle comes out automatically after valid authentication or other validation.

8. A medical injector according to claim 1, characterized in that for authentication a biometric sensor like a finger print reading sensor is applied.

9. A medical injector according to claim 1, characterized in that for authentication a code generating interface like a number terminal is applied.

10. A medical injector according to claim 1, characterized in that the medical injector is a common portable unit.

11. A medical injector according to claim 1, characterized in that the medical injector is a stationary unit.

12. A medical injector according to claim 1, characterized in that a data transfer and/or programming interface (8) is applied to the injector-unit.

13. A medical injector according to claim 12, characterized in that the injector is also embodied with a data or data network transfer interface with a two-way-data-transfer in order to communicate use data as well as therapy and patient data internally and externally with a computer or computer system or computer network.

14. A medical injector according to claim 13, characterized in that the injector is applied with internal storage means or can correspond with external storage means, in order to result validation of use, or will give actual usage information.

15. A medical injector according to claim 13, characterized in that patient allergy data for various drugs is stored internally or externally in that way, that the injector will be deactivated automatically, when the drug is not suitable with the patient.

16. A medical injector, according to claim 13, characterized in that if the current drug reached a programmable filling state mark, for example almost nearly completion, then a signal generates automatically an order of newer drug, if authorized by the physician.

17. A medical injector according to claim 1, characterized in that a timer (7) and/or an alarm facility (9) is implemented into the injector-unit.

18. A medical injector according to claim 17, characterized in that if a timer is implemented, the timer is applied with an Auto-Off-function, in order to switch off the injector if the validation of authentication will not be given in an adjustable time limit.

19. A medical injector according to claim 1, characterized in that a compact lithium cell is implemented into the injector, or injector-unit.
